(19) United States
(12) Patent Application Publication
(10) Pub. No.: US 2023/0111768 A1
(43) Pub. Date: Apr. 13, 2023

(54) METHOD FOR SYNTHESIZING AN ANTI-CANCER COMPOUND THROUGH A ONE-POT REACTION

(71) Applicants: Nasim Batooie, Kermanshah (IR); Mohammad Mehdi Khodaei, Kermanshah (IR); Kiumars Bahrami, Kermanshah (IR); Seyyed Shahram Miraghaee, Kermanshah (IR)

(72) Inventors: Nasim Batooie, Kermanshah (IR); Mohammad Mehdi Khodaei, Kermanshah (IR); Kiumars Bahrami, Kermanshah (IR); Seyyed Shahram Miraghaee, Kermanshah (IR)

(73) Assignees: Nasim Batooie, Kermansh (IR); Mohammad Mehdi Khodaei, Kermanshah (IR); Kiumars Bahrami, Kermanshah (IR); Seyyed Shahram Miraghaee, Kermanshah (IR); Razi University, Kermanshah (IR)

(21) Appl. No.: 18/080,744

(22) Filed: Dec. 14, 2022

Related U.S. Application Data
(60) Provisional application No. 63/289,158, filed on Dec. 14, 2021.

(51) Int. Cl. C07D 513/14; C07D 513/04

(52) U.S. Cl. CPC C07D 513/14; C07D 513/04

(57) ABSTRACT

A method for synthesizing a compound comprising forming a liquid mixture by adding a CH-acid, 2-Mercaptobenzimidazole, and a benzaldehyde component to an ionic liquid of trihexyltetradecylphosphonium bromide. The method may further comprise mixing the liquid mixture at a temperature between 20° C. and 30° C. for a time duration between 17 and 30 minutes, and forming the compound by adding an ethanol-water solution to the mixed liquid mixture with a volume ratio (ethanol-water solution:mixed liquid mixture) between 1:1 and 1.5:1.

### CROSS-REFERENCE TO RELATED APPLICATION

This application claims the benefit of priority from U.S. Provisional Patent Application Ser. No. 63/289,158, filed on Dec. 14, 2021, entitled “ONE-POT SYNTHESIS OF BENZO[4,5]IMIDAZO[2,1-B][1,3] THIAZIN-4-ONE” which is incorporated herein by reference in its entirety.

### TECHNICAL FIELD

The present disclosure is generally related to an exemplary method for synthesizing an anti-cancer compound comprising a benzimidazole part, a pyrimidine part, and a thiazine part; and more particularly to an exemplary method for synthesizing benzo[4,5]imidazo[2,1-b]pyrimido[4,5-d][1,3]thiazine-2,4(3H)-dione and benzo[4,5]imidazo[2,1-b][1,3]thiazin-4-one through a one-pot reaction.

### BACKGROUND

In recent decades, growth of global population (especially aging population) in addition to the increase of different risk factors, such as tendency to eating unhealthy foods, sedentary lifestyle, obesity, diabetes, etc., have led to a dramatic rise in cancer cases. According to the World Health Organization, cancer is the second cause of death globally and may soon overtake cardiovascular diseases that rank first in global causes of death.

In spite of the wide variety of chemotherapy drugs for cancer treatment, many patients may exhibit a poor response to some chemotherapy drugs due to the development of resistance or poor efficacy. Thus, there is an urgent need for developing novel and effective anti-cancer drugs using manufacturing processes with improved efficiency, decreased reaction time, more environment-friendly reaction procedure, and less reactant consumption.

### SUMMARY

This summary is intended to provide an overview of the subject matter of the present disclosure, and is not intended to identify essential elements or key elements of the subject matter, nor is it intended to be used to determine the scope of the claimed implementations. Its sole purpose is to present some concepts of one or more exemplary aspects in a simplified form as a prelude to the more detailed description that is presented later. The proper scope of the present disclosure may be ascertained from the claims set forth below in view of the detailed description below and the drawings.

One or more exemplary embodiments describe an exemplary method for synthesizing an exemplary compound of the following structural formula (I).

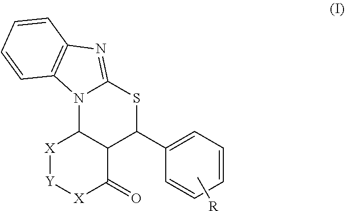

In an exemplary embodiment, X may be selected from an exemplary group consisting of NCH₃ and CH₂, Y may be selected from an exemplary group consisting of C═O and C(CH₃)₂, and R may be selected from an exemplary group consisting of 4-CH₃ and 4-NO₂. In an exemplary embodiment, an exemplary method may comprise forming an exemplary liquid mixture by adding an exemplary CH-acid, 2-Mercaptobenzimidazole, and an exemplary benzaldehyde component to an exemplary ionic liquid of trihexyltetradecylphosphonium bromide. In an exemplary embodiment CH-acid may be selected from an exemplary group consisting of 1,3-Dimethylbarbituric acid and Dimedone, and an exemplary benzaldehyde component may be selected from an exemplary group consisting of 4-Methylbenzaldehyde and 4-Nitrobenzaldehyde.

In an exemplary embodiment, an exemplary method may further comprise mixing an exemplary liquid mixture at an exemplary temperature between 20° C. and 30° C. for an exemplary time duration between 17 and 30 minutes. In an exemplary embodiment, an exemplary method may further comprise forming an exemplary compound by adding an exemplary ethanol-water solution to an exemplary mixed liquid mixture with an exemplary volume ratio (ethanol-water solution:mixed liquid mixture) between 1:1 and 1.5:1. In an exemplary embodiment, an exemplary ethanol-water solution may comprise ethanol with an exemplary concentration between 30% (v/v) and 45% (v/v).

This Summary may introduce a number of concepts in a simplified format; the concepts are further disclosed within the “Detailed Description” section. This Summary is not intended to configure essential/key features of the claimed subject matter, nor is intended to limit the scope of the claimed subject matter.

### BRIEF DESCRIPTION OF THE DRAWINGS

The novel features which are believed to be characteristic of the present disclosure, as to its structure, organization, use and method of operation, together with further objectives and advantages thereof, will be better understood from the following drawings in which an exemplary embodiment will now be illustrated by way of example. It is expressly understood, however, that the drawings are for the purpose of illustration and description only and are not intended as a definition of the limits of the present disclosure. Exemplary embodiments will now be described by way of example in association with the accompanying drawings in which:

FIG. 1 illustrates an exemplary flowchart of an exemplary method for synthesizing an exemplary compound of general formula (I), consistent with one or more exemplary embodiments of the present disclosure;

FIG. 2 illustrates an exemplary flowchart of an exemplary method for forming an exemplary liquid mixture, consistent with one or more exemplary embodiments of the present disclosure;

FIG. 3 illustrates an exemplary reaction scheme for forming an exemplary ionic liquid of trihexyltetradecylphosphonium bromide, consistent with one or more exemplary embodiments of the present disclosure;

FIG. 4 illustrates an exemplary reaction scheme for forming an exemplary compound of general formula (I), consistent with one or more exemplary embodiments of the present disclosure;

FIG. 5 illustrates an exemplary reaction scheme for forming benzo[4,5]imidazo[2,1-b]pyrimido[4,5-d][1,3]thiazine-2,4(3H)-dione, consistent with one or more exemplary embodiments of the present disclosure;

FIG. 6 illustrates an exemplary reaction scheme for forming benzo[4,5]imidazo[2,1-b][1,3]thiazin-4-one, consistent with one or more exemplary embodiments of the present disclosure;

FIG. 7 illustrates an exemplary schematic representation of a mechanism for forming an exemplary compound of general formula (I), consistent with one or more exemplary embodiments of the present disclosure;

FIG. 8A illustrates Fourier-Transform Infrared (FTIR) spectrum of benzo[4,5]imidazo[2,1-b]pyrimido[4,5-d][1,3]thiazine-2,4(3H)-dione, consistent with one or more exemplary embodiments of the present disclosure;

FIG. 8B illustrates FTIR spectrum of benzo[4,5]imidazo[2,1-b][1,3]thiazin one, consistent with one or more exemplary embodiments of the present disclosure;

FIG. 9A illustrates an exemplary proton nuclear magnetic resonance CH NMR) spectrum of benzo[4,5]imidazo[2,1-b]pyrimido[4,5-d][1,3]thiazine-2,4(3H)-dione, consistent with one or more exemplary embodiments of the present disclosure;

FIG. 9B illustrates an exemplary ¹H NMR spectrum of benzo[4,5]imidazo[2,1-b][1,3]thiazin-4-one, consistent with one or more exemplary embodiments of the present disclosure;

FIG. 10 illustrates charts of cell survival assessment after 24 hours of treatment with benzo[4,5]imidazo[2,1-b][1,3]thiazin-4-one, consistent with one or more exemplary embodiments of the present disclosure; and

FIG. 11 illustrates charts of cell survival assessment after 24 hours of treatment with benzo[4,5]imidazo[2,1-b]pyrimido[4,5-d][1,3]thiazine-2,4(3H)-dione, consistent with one or more exemplary embodiments of the present disclosure.

### DETAILED DESCRIPTION

In the following detailed description, numerous specific details are set forth by way of examples to provide a thorough understanding of the relevant teachings related to the exemplary embodiments. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in one or more exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be plain to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Disclosed herein is an exemplary method for synthesizing an exemplary compound comprising the following general formula (I) through an exemplary one-pot reaction.

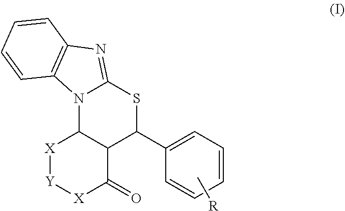

In an exemplary embodiment, with respect to general formula (I), X may be selected from an exemplary group consisting of NCH₃ and CH₂, Y may be selected from an exemplary group consisting of C═O and C(CH₃)₂, and R may be selected from an exemplary group consisting of 4-CH₃ and 4-NO₂. In an exemplary embodiment, an exemplary compound may have the following structural formula (II) with an International Union of Pure and Applied Chemistry (IUPAC) name benzo[4,5]imidazo[2,1-b]pyrimido[4,5-d][1,3]thiazine-2,4(3H)-dione and IUPAC formula of C₂₁H₁₈N₄O₂S.

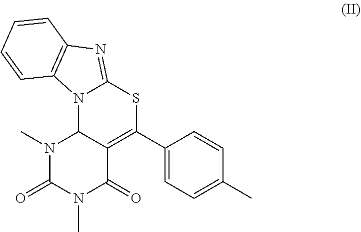 [SMILES: Cc1ccc(C2=C3C(=O)N(C)C(=O)N(C)C3n3c(nc4ccccc43)S2)cc1]

In an exemplary embodiment, an exemplary compound may have the following structural formula (III) with an IUPAC name benzo[4,5]imidazo[2,1-b][1,3]thiazin-4-one and IUPAC formula of C₂₂H₁₉N₃O₃S.

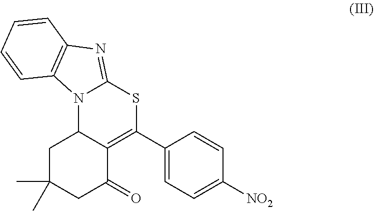 [SMILES: CC1(C)CC(=O)C2=C(c3ccc([N+](=O)[O-])cc3)Sc3nc4ccccc4n3C2C1]

An exemplary compound of general formula (I) may have an anti-cancer effect on cancer cells including, but not limited to, skin cancer cells, prostate cancer cells, and pancreas cancer cells, due to having a benzimidazole part, a pyrimidine part, and a thiazine part. In an exemplary embodiment, an exemplary method for synthesizing an exemplary compound may include synthesizing an exemplary compound of general formula (I) in an exemplary one-pot reaction. One-pot reaction may refer to a reaction in which all exemplary reactants may be subject to consecutive chemical reactions in a same reactor. An exemplary one-pot reaction may be much straightforward and may prevent lengthy separation and purification processes of intermediate compounds. Thus, an exemplary one-pot reaction may improve efficiency of an exemplary chemical reaction, save resources and time, and result in a high chemical yield.

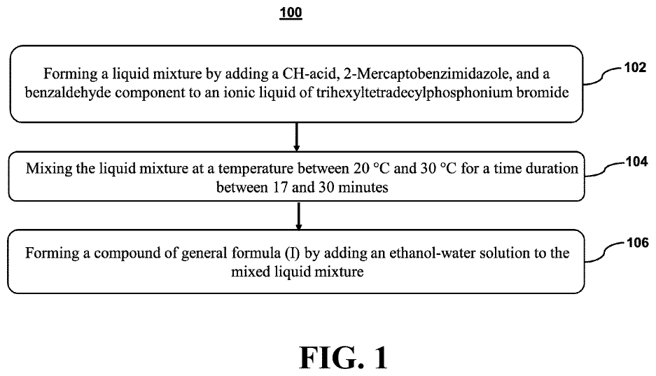

Referring to the figures, FIG. 1 illustrates an exemplary flowchart of exemplary method 100 for synthesizing an exemplary compound of general formula (I), consistent with one or more exemplary embodiments of the present disclosure. In an exemplary embodiment, exemplary method 100 may include: forming an exemplary liquid mixture by adding an exemplary CH-acid, 2-Mercaptobenzimidazole (C₇H₆N₂S), and an exemplary benzaldehyde component to an exemplary ionic liquid of trihexyltetradecylphosphonium bromide (C₃₂H₆₈BrP) (step 102); mixing an exemplary liquid mixture at an exemplary temperature between 20° C. and 30° C. for an exemplary time duration between 17 and 30 minutes (step 104); and forming an exemplary compound of general formula (I) by adding an exemplary ethanol-water solution to an exemplary mixed liquid mixture (step 106).

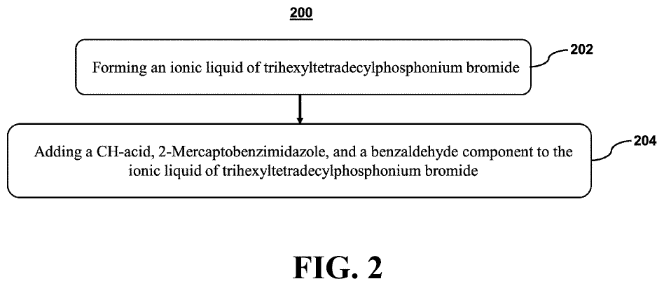

In further detail with respect to step 102, step 102 may include forming an exemplary liquid mixture by adding an exemplary CH-acid, 2-Mercaptobenzimidazole, and an exemplary benzaldehyde component to an exemplary ionic liquid of trihexyltetradecylphosphonium bromide. In an exemplary embodiment, details of step 102 for forming an exemplary liquid mixture are described in FIG. 2. FIG. 2 illustrates an exemplary flowchart of exemplary method 200 for forming an exemplary liquid mixture, consistent with one or more exemplary embodiments of the present disclosure. Accordingly, exemplary method 200 may be similar to exemplary step 102. In an exemplary embodiment, exemplary method 200 may include forming an exemplary ionic liquid of trihexyltetradecylphosphonium bromide (step 202), and adding an exemplary CH-acid, 2-Mercaptobenzimidazole, and an exemplary benzaldehyde component to an exemplary ionic liquid of trihexyltetradecylphosphonium bromide (step 204).

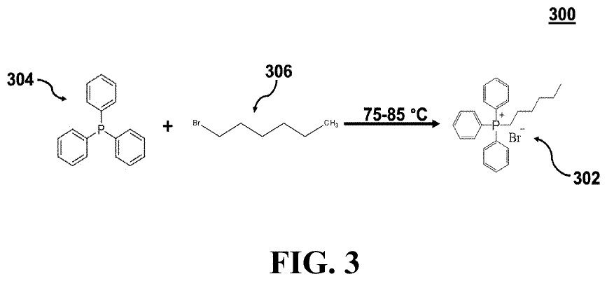

In further detail with respect to step 202, step 202 may include forming an exemplary ionic liquid of trihexyltetradecylphosphonium bromide. In an exemplary embodiment, forming an exemplary ionic liquid of trihexyltetradecylphosphonium bromide may include forming an exemplary ionic liquid comprising trihexyltetradecylphosphonium bromide with a concentration between about 45% (w/w) and 55% (w/w). In an exemplary embodiment, an exemplary ionic liquid of trihexyltetradecylphosphonium may be produced by reacting Triphenylphosphine with Bromohexane in an exemplary laboratory container including, but not limited to, beakers, tins, flasks, bottles, buckets, basins, bowls, vials, tubes, barrels, cannisters, etc. In an exemplary embodiment, details of step 202 for reacting Triphenylphosphine with Bromohexane are described in context of elements presented in FIG. 3. FIG. 3 illustrates exemplary reaction scheme 300 for forming an exemplary ionic liquid of trihexyltetradecylphosphonium bromide 302, consistent with one or more exemplary embodiments of the present disclosure. In further detail with respect to exemplary reaction scheme 300, an exemplary ionic liquid of trihexyltetradecylphosphonium bromide 302 may be formed by reacting Triphenylphosphine 304 with Bromohexane 306 at an exemplary temperature ranging between about 75° C. and 85° C. In an exemplary embodiment, Triphenylphosphine 304 may be reacted with Bromohexane 306 in ethanol as an exemplary solvent. In an exemplary embodiment, an exemplary ionic liquid of trihexyltetradecylphosphonium bromide may be prepared by mixing Triphenylphosphine (e.g., in form of solid) and Bromohexane (e.g., in form of liquid) with an ethanol solution (e.g., an ethanol solution with a concentration of at least 95% (v/v)) on a stirrer (e.g., a magnetic stirrer) at a temperature level of about 80° C. for a time duration of about 3 hours. In an exemplary embodiment, Triphenylphosphine and Bromohexane may be added to an exemplary ethanol solution (95% (v/v) or more) with a weight ratio (Triphenylphosphine:Bromohexane) of about 1:1. For example, in an exemplary embodiment, to prepare about 10 mL of an exemplary ionic liquid of trihexyltetradecylphosphonium, about 1 mmol of Triphenylphosphine and about 1 mmol of Bromohexane may be added to about 10 mL ethanol solution (e.g., with a concentration of at least 95% (v/v)), in an exemplary laboratory container (e.g., a beaker, flask, etc.), and may be stirred—using a magnetic stirrer—at about 80° C. for about 3 hours.

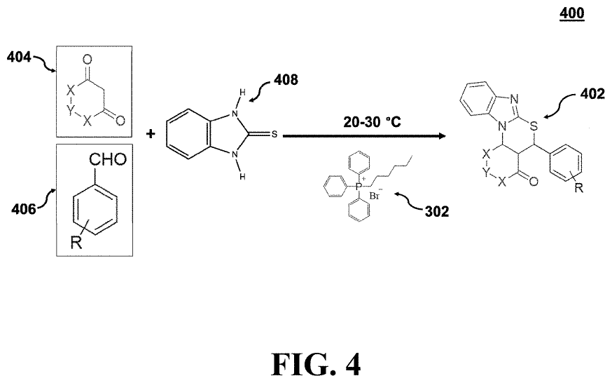

In further detail with respect to step 204, step 204 may include adding an exemplary CH-acid, 2-Mercaptobenzimidazole, and an exemplary benzaldehyde component to an exemplary ionic liquid of trihexyltetradecylphosphonium bromide. In an exemplary embodiment, adding an exemplary CH-acid, 2-Mercaptobenzimidazole, and an exemplary benzaldehyde component to an exemplary ionic liquid of trihexyltetradecylphosphonium bromide may include adding an exemplary CH-acid (e.g., in form of solid), 2-Mercaptobenzimidazole (e.g., in form of solid), and an exemplary benzaldehyde component (e.g., in form of liquid) to an exemplary ionic liquid of trihexyltetradecylphosphonium bromide—in an exemplary laboratory container (e.g., a beaker)—such that an exemplary molar ratio of CH-acid, 2-Mercaptobenzimidazole, benzaldehyde component, and trihexyltetradecylphosphonium bromide in an exemplary final liquid mixture may become about 1:1:1:1:1. In an exemplary embodiment, adding an exemplary CH-acid, 2-Mercaptobenzimidazole, and an exemplary benzaldehyde component to an exemplary ionic liquid of trihexyltetradecylphosphonium bromide may include adding about 0.8-1.2 mmol of an exemplary CH-acid, about 0.8-1.2 mmol 2-Mercaptobenzimidazole, and about 0.8-1.2 mmol of an exemplary benzaldehyde component to an exemplary ionic liquid of trihexyltetradecylphosphonium bromide containing about 0.8-1.2 mmol trihexyltetradecylphosphonium bromide, in an exemplary laboratory container (e.g., a beaker). Exemplary precursors including an exemplary CH-acid, 2-Mercaptobenzimidazole, and an exemplary benzaldehyde component may be added to an exemplary ionic liquid of trihexyltetradecylphosphonium bromide using a spatula, a graduated cylinder, a pipet, etc. In an exemplary embodiment, details of a general reaction between an exemplary CH-acid, 2-Mercaptobenzimidazole, and an exemplary benzaldehyde component set forth in step 102 and step 204 is described in context of elements presented in FIG. 4. FIG. 4 illustrates exemplary reaction scheme 400 for forming an exemplary compound of general formula (I) 402, consistent with one or more exemplary embodiments of the present disclosure. In an exemplary embodiment, exemplary reaction scheme 400 may include exemplary precursors including exemplary CH-acid 404, exemplary benzaldehyde component 406, 2-Mercaptobenzimidazole 408, and trihexyltetradecylphosphonium bromide 302.

Furthermore, in an exemplary embodiment, an exemplary CH-acid may be selected from an exemplary group consisting of 1,3-Dimethylbarbituric acid (C₆H₈N₂₀₃) and Dimedone (C₈H₁₂₀₂), and an exemplary benzaldehyde component may be selected from an exemplary group consisting of 4-Methylbenzaldehyde (C₈H₈₀) and 4-Nitrobenzaldehyde (C₇H₅NO₃). For example, in case of synthesizing an exemplary compound of structural formula (II) (i.e., benzo[4,5]imidazo[2,1-b]pyrimido[4,5-d][1,3]thiazine-2,4(3H)-dione), adding an exemplary CH-acid, 2-Mercaptobenzimidazole, and an exemplary benzaldehyde component to an exemplary ionic liquid of trihexyltetradecylphosphonium bromide may include adding 1,3-Dimethylbarbituric acid, 2-Mercaptobenzimidazole, and 4-Methylbenzaldehyde (e.g., using a spatula, a graduated cylinder, a pipet, etc.) to an exemplary ionic liquid of trihexyltetradecylphosphonium bromide. In an exemplary embodiment, adding 1,3-Dimethylbarbituric acid, 2-Mercaptobenzimidazole, and 4-Methylbenzaldehyde to an exemplary ionic liquid of trihexyltetradecylphosphonium bromide may include adding about 0.8-1.2 mmol of 1,3-Dimethylbarbituric acid, about 0.8-1.2 mmol 2-Mercaptobenzimidazole, and about 0.8-1.2 mmol 4-Methylbenzaldehyde to an exemplary ionic liquid of trihexyltetradecylphosphonium bromide containing about 0.8-1.2 mmol trihexyltetradecylphosphonium bromide, in an exemplary laboratory container (e.g., a beaker).

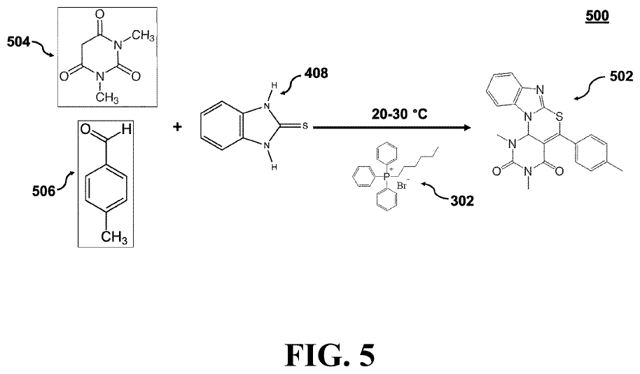

In an exemplary embodiment, details of an exemplary reaction between 1,3-Dimethylbarbituric acid, 2-Mercaptobenzimidazole, and 4-Methylbenzaldehyde set forth in step 204 is described in context of elements presented in FIG. 5. FIG. 5 illustrates exemplary reaction scheme 500 for forming benzo[4,5]imidazo[2,1-b]pyrimido[4,5-d][1,3]thiazine-2,4(3H)-dione 502, consistent with one or more exemplary embodiments of the present disclosure. In an exemplary embodiment, exemplary reaction scheme 500 may include exemplary precursors including 1,3-Dimethylbarbituric acid 504, 4-Methylbenzaldehyde 506, 2-Mercaptobenzimidazole 408, and trihexyltetradecylphosphonium bromide 302.

In an exemplary embodiment, in case of synthesizing an exemplary compound of structural formula (III) (i.e., benzo[4,5]imidazo[2,1-b][1,3]thiazin-4-one), adding an exemplary CH-acid, 2-Mercaptobenzimidazole, and an exemplary benzaldehyde component to an exemplary ionic liquid of trihexyltetradecylphosphonium bromide may include adding Dimedone (also known as 5,5-Dimethylcyclohexane-1,3-dione), 2-Mercaptobenzimidazole, and 4-Nitrobenzaldehyde (e.g., using a spatula, a graduated cylinder, a pipet, etc.) to an exemplary ionic liquid of trihexyltetradecylphosphonium bromide. In an exemplary embodiment, adding Dimedone, 2-Mercaptobenzimidazole, and 4-Nitrobenzaldehyde to an exemplary ionic liquid of trihexyltetradecylphosphonium bromide may include adding about 0.8-1.2 mmol Dimedone, about 0.8-1.2 mmol 2-Mercaptobenzimidazole, and about 0.8-1.2 mmol 4-Nitrobenzaldehyde to an exemplary ionic liquid of trihexyltetradecylphosphonium bromide containing about 0.8-1.2 mmol trihexyltetradecylphosphonium bromide, in an exemplary laboratory container (e.g., a beaker).

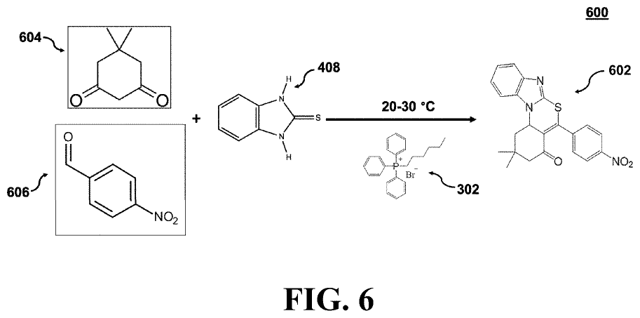

In an exemplary embodiment, details of an exemplary reaction between Dimedone, 2-Mercaptobenzimidazole, and 4-Nitrobenzaldehyde set forth in step 204 is described in context of elements presented in FIG. 6. FIG. 6 illustrates exemplary reaction scheme 600 for forming benzo[4,5]imidazo[2,1-b][1,3]thiazin-4-one 602, consistent with one or more exemplary embodiments of the present disclosure. In an exemplary embodiment, exemplary reaction scheme 600 may include exemplary precursors including Dimedone 604, 4-Nitrobenzaldehyde 606, 2-Mercaptobenzimidazole 408, and trihexyltetradecylphosphonium bromide 302.

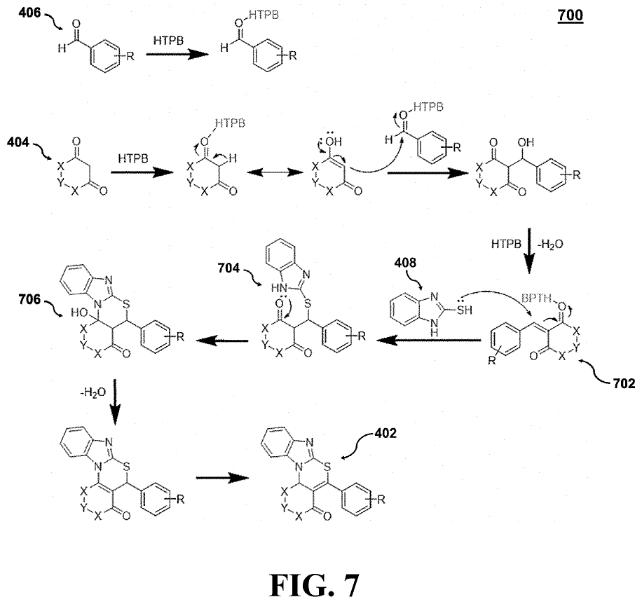

In further detail with respect to step 104, step 104 may include mixing an exemplary liquid mixture (i.e., an exemplary liquid mixture set forth in step 102) at an exemplary temperature between about 20° C. and 30° C. for an exemplary time duration between about 17 and 30 minutes. In an exemplary embodiment, mixing an exemplary liquid mixture at an exemplary temperature between about 20° C. and 30° C. for an exemplary time duration between about 17 and 30 minutes may include stirring/mixing (e.g., using a magnetic stirring) an exemplary liquid mixture for a time duration between about 20 and 25 minutes (e.g., 20 minutes) while an exemplary temperature of an exemplary liquid mixture is maintained at an exemplary temperature ranging between 20° C. and 25° C. (for example by placing an exemplary container of an exemplary liquid mixture in an stirrer incubator that is adjusted to a temperature ranging between 20° C. and 25° C.). In an exemplary embodiment, an exemplary liquid mixture may comprise trihexyltetradecylphosphonium bromide with a concentration between about 47% (w/w) and 53% (w/w), an exemplary CH-acid (i.e., 1,3-Dimethylbarbituric acid or Dimedone) with a concentration between about 15.3% (w/w) and 21.3% (w/w), 2-Mercaptobenzimidazole with a concentration between about 14.5% (w/w) and 20.6% (w/w), and an exemplary benzaldehyde component (i.e., 4-Methylbenzaldehyde or 4-Nitrobenzaldehyde) with a concentration between about 11.5% (w/w) and 17.5% (w/w). In an exemplary embodiment, details of a reaction mechanism that may occur during mixing an exemplary liquid mixture in step 104 is described in context of elements presented in FIG. 7.

FIG. 7 illustrates exemplary schematic representation of mechanism 700 for forming an exemplary compound of general formula (I) 402, consistent with one or more exemplary embodiments of the present disclosure. In further detail with respect to mechanism 700, trihexyltetradecylphosphonium bromide (shown as HTPB in FIG. 7) may have an exemplary phosphorus atom that is a positive ion and may have a strong tendency to interact with an exemplary oxygen atom of an exemplary carbonyl group (as a donor) to produce an exemplary phosphorus atom with 5 bonds. Thus, HTPB may activate an exemplary carbonyl group in both of exemplary CH-acid 404 and exemplary benzaldehyde component 406 that may result in an exemplary Knoevenagel condensation between exemplary CH-acid 404 and exemplary benzaldehyde component 406. Condensation of exemplary CH-acid 404 and exemplary benzaldehyde component 406 may form an exemplary α,β-unsaturated carbonyl compound 702. “Knoevenagel condensation” may refer to a nucleophilic addition of an aldehyde or ketone to an activated methylene compound using an amine base (such as piperidine or pyridine) as a catalyst followed by a dehydration reaction in which a molecule of water (H₂O) may be eliminated (as a result of condensation). Furthermore, exemplary intermediate 704 may be formed through an exemplary Michael addition reaction between α,β-unsaturated carbonyl compound 702 and 2-Mercaptobenzimidazole 408. “Michael addition reaction” may refer to a nucleophilic addition reaction comprising addition of a carbanion (or an exemplary nucleophile) to an α,β-unsaturated carbonyl compound that may have an exemplary functional group with electron-withdrawing feature. Exemplary intermediate 704 may be converted to exemplary intermediate 706 through an exemplary cyclization reaction. “Cyclization reaction” may refer to a reaction that may be initiated by formation of a putative cation either by ionization or by electrophilic addition to a double bond, e.g., from a sp³-hybridized carbon. In an exemplary embodiment, exemplary intermediate 706 may be converted to exemplary compound of general formula (I) 402 by losing a H₂O molecule. In an exemplary embodiment, exemplary compound of general formula (I) 402 may include exemplary compound of structural formula (II) 502 (see FIG. 5) and exemplary compound of structural formula (III) 602 (see FIG. 6). In further detail with respect to FIG. 7, X may be selected from an exemplary group consisting of NCH₃ and CH₂, Y may be selected from an exemplary group consisting of C═O and C(CH₃)₂, and R may be selected from an exemplary group consisting of 4-CH₃ and 4-NO₂.

In further detail with respect to step 106, step 106 may include forming an exemplary compound of general formula (I) by adding an exemplary ethanol-water solution to an exemplary mixed liquid mixture. In an exemplary embodiment, forming an exemplary compound of general formula (I) may include forming an exemplary compound of general formula (I) in form of crystalline solids. In an exemplary embodiment, adding an exemplary ethanol-water solution to an exemplary mixed liquid mixture may include adding an exemplary ethanol-water solution to an exemplary mixed liquid mixture with a volume ratio (ethanol-water solution:mixed liquid mixture) between about 1:1 and 1.5:1. In an exemplary embodiment, an exemplary ethanol-water solution may comprise ethanol with a concentration between about 30% (v/v) and 45% (v/v). In an exemplary embodiment, an exemplary water used in ethanol-water solution may include distilled water, double-distilled water, or ultrapure water. “Ultrapure water” may refer to a water that has been purified using a combination of ultrafiltration technologies and ultraviolet photo-oxidation system. An ultrapure water may be RNase-free and/or pyrogen-free, and may be ultra-low in organics. In an exemplary embodiment, adding an exemplary ethanol-water solution to an exemplary mixed liquid mixture may include adding an exemplary ethanol-water solution (containing about 30-45% (v/v) ethanol) to an exemplary mixed liquid mixture with a volume ratio (ethanol-water solution:mixed liquid mixture) of about 1:1 (e.g., adding about 10 mL ethanol-water solution to about 10 mL of an exemplary mixed liquid mixture).

In an exemplary embodiment, exemplary crystalline solids of an exemplary compound of general formula (I), formed in step 106, may be isolated by filtering the resulting mixture of ethanol-water solution and an exemplary mixed liquid mixture through a filter paper. For example, in case of synthesizing benzo[4,5]imidazo[2,1-b]pyrimido[4,5-d][1,3]thiazine-2,4(3H)-dione, exemplary crystalline solids of benzo[4,5]imidazo[2,1-b]pyrimido[4,5-d][1,3]thiazine-2,4(3H)-dione may be isolated by filtering the resulting mixture of ethanol-water solution and an exemplary mixed liquid mixture through a filter paper. Meanwhile, in case of synthesizing benzo[4,5]imidazo[2,1-b][1,3]thiazin-4-one, exemplary crystalline solids of benzo[4,5]imidazo[2,1-b][1,3]thiazin-4-one may be isolated by filtering the resulting mixture of ethanol-water solution and an exemplary mixed liquid mixture through a filter paper.

In an exemplary embodiment, one or more exemplary chromatographic techniques may be used to purify an exemplary compound of general formula (I) formed in step 106 (e.g., benzo[4,5]imidazo[2,1-b]pyrimido[4,5-d][1,3]thiazine-2,4(3H)-dione or benzo[4,5]imidazo[2,1-b][1,3]thiazin-4-one) and form a highly purified compound of general formula (I). Exemplary chromatographic techniques may include, but are not limited to, thin layer chromatography (TLC) and/or column chromatography. In an exemplary embodiment, an exemplary compound of general formula (I) may be isolated through an exemplary silica gel TLC, followed by recrystallization. For example, in an exemplary embodiment, the formed exemplary compound of general formula (I) in step 106 may be dissolved in ethyl acetate and dropped onto an exemplary Silica TLC Plate (e.g., a glass-backed TLC plate with 250-μm thickness and 20×20 cm dimensions). In an exemplary embodiment, an exemplary solvent mix of hexane:ethyl acetate with a molar ratio of about 4:1 may be used as an exemplary mobile phase in TLC of an exemplary compound of general formula (I). In an exemplary embodiment, an exemplary silica gel TLC may result in the formation of a plurality of bands on an exemplary TLC plate including, but not limited to, a CH-acid band (e.g., a 1,3-Dimethylbarbituric acid band or a Dimedone band), a 2-Mercaptobenzimidazole band, a benzaldehyde component band (e.g., a 4-Methylbenzaldehyde band or a 4-Nitrobenzaldehyde band), and a band of an exemplary compound of general formula (I) (e.g., a benzo[4,5]imidazo[2,1-b]pyrimido[4,5-d][1,3]thiazine-2,4(3H)-dione band, or a benzo[4,5]imidazo[2,1-b][1,3]thiazin-4-one band). An exemplary band of compound of general formula (I) (e.g., a benzo[4,5]imidazo[2,1-b]pyrimido[4,5-d][1,3]thiazine-2,4(3H)-dione band or a benzo[4,5]imidazo[2,1-b][1,3]thiazin-4-one band) may be scraped from an exemplary TLC plate. An exemplary scraped band may then be covered by ethyl acetate (e.g., in a plate) while stirring using a stirrer, e.g., a magnetic stirrer. Mixture of an exemplary scraped band and ethyl acetate may, subsequently, be filtered and ethyl acetate may be removed by rotary evaporation, resulting in precipitation of a solid that may comprise an exemplary compound of formula (I). In an exemplary embodiment, an exemplary TLC may result in the formation of an exemplary compound of general formula (I) (e.g., benzo[4,5]imidazo[2,1-b]pyrimido[4,5-d][1,3]thiazine-2,4(3H)-dione or benzo[4,5]imidazo[2,1-b][1,3]thiazin-4-one) with at least 90% purity. In one or more exemplary embodiments, an exemplary compound of general formula (I) may be produced in different exemplary dosage forms, including but not limited to, tablets, capsules, geltabs, powder, granules, solution, and/or suspension.

### Examples

Hereinafter, one or more exemplary embodiments will be described in further detail with reference to examples. It will be obvious to a person having ordinary skill in the art that these examples may be for illustrative purposes only and are not to be interpreted to limit the scope of the present disclosure.

### Example 1: Spectroscopic Analysis of benzo[4,5]imidazo[2,1-b]pyrimido[4,5-d][1,3]thiazine-2,4(3H)-dione and benzo[4,5]imidazo[2,1-b][1,3]thiazin-4-one

In this example physical properties of benzo[4,5]imidazo[2,1-b]pyrimido[4,5-d][1,3]thiazine-2,4(3H)-dione (i.e., an exemplary compound of structural formula (II)) and benzo[4,5]imidazo[2,1-b][1,3]thiazin-4-one (i.e., an exemplary compound of structural formula (III)) was investigated by Fourier-Transform Infrared (FTIR) and proton nuclear magnetic resonance ¹H NMR).

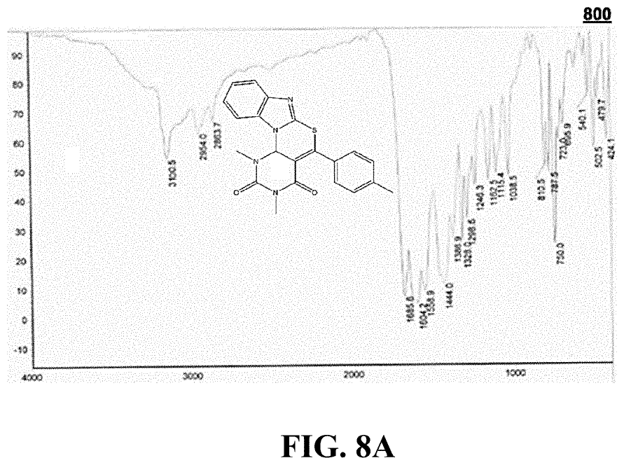

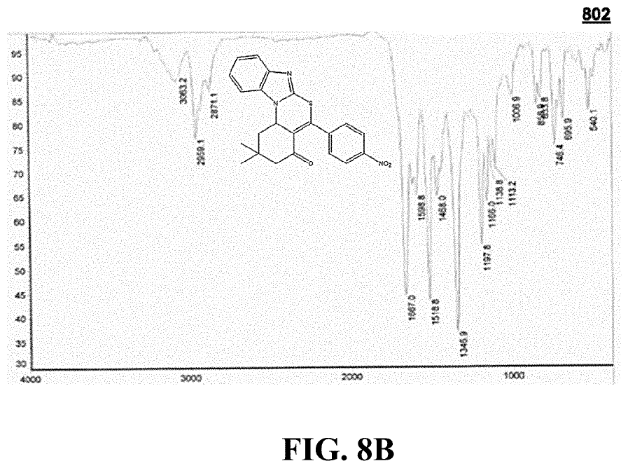

FIG. 8A illustrates FTIR spectrum 800 of benzo[4,5]imidazo[2,1-b]pyrimido[4,5-d][1,3]thiazine-2,4(3H)-dione, consistent with one or more exemplary embodiments of the present disclosure. FTIR analysis may be used to characterize and confirm the formation of benzo[4,5]imidazo[2,1-b]pyrimido[4,5-d][1,3]thiazine-2,4(3H)-dione (i.e., an exemplary compound of structural formula (II)). In further detail with respect to FTIR spectrum 800, FTIR spectrum 800 may exhibit peaks at about 3100.5 cm⁻¹ and 1685.6 cm¹ which may indicate the presence of C—H stretching and carbonyl group in an exemplary structure of benzo[4,5]imidazo[2,1-b]pyrimido[4,5-d][1,3]thiazine-2,4(3H)-dione. Two strong peaks were detected at wavelengths of about 1604.2 and 1558.9 cm¹ that may indicate the presence of C═C and C═N stretching, respectively. Meanwhile, 2 C—N stretching were observed at wavelengths of about 1388.9 and 1328.0 cm¹.

FIG. 8B illustrates FTIR spectrum 802 of benzo[4,5]imidazo[2,1-b][1,3]thiazin-4-one, consistent with one or more exemplary embodiments of the present disclosure. FTIR analysis may be used to characterize and confirm the formation of benzo[4,5]imidazo[2,1-b)][1,3]thiazin-4-one (i.e., an exemplary compound of structural formula (III)). In further detail with respect to FTIR spectrum 802, FTIR spectrum 802 illustrates peaks at about 3063.2 cm⁻¹ which may indicate the presence of a C—H stretching in an exemplary structure of benzo[4,5]imidazo[2,1-b][1,3]thiazin-4-one. Peaks at 1667 cm¹ and 1598.8 cm¹ are both characteristics of C═C stretching. Meanwhile, peaks detected at about 1518.8 cm¹ and 1346.9 cm¹ may indicate the presence of a N—O stretching and O—H bending, respectively, in an exemplary structure of benzo[4,5]imidazo[2,1-b][1,3]thiazin-4-one.

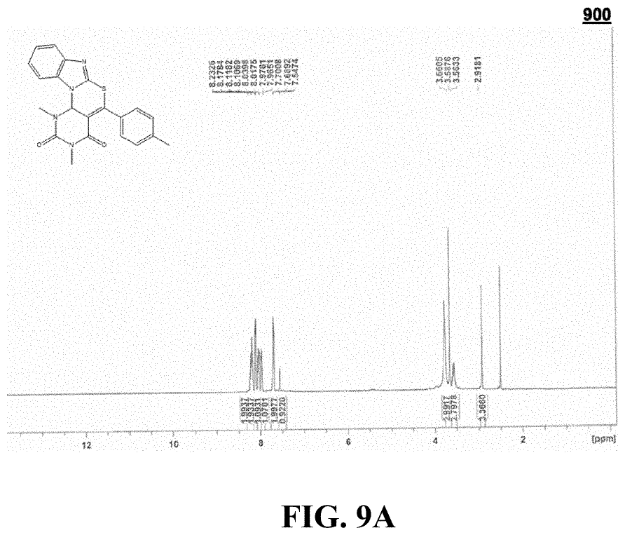

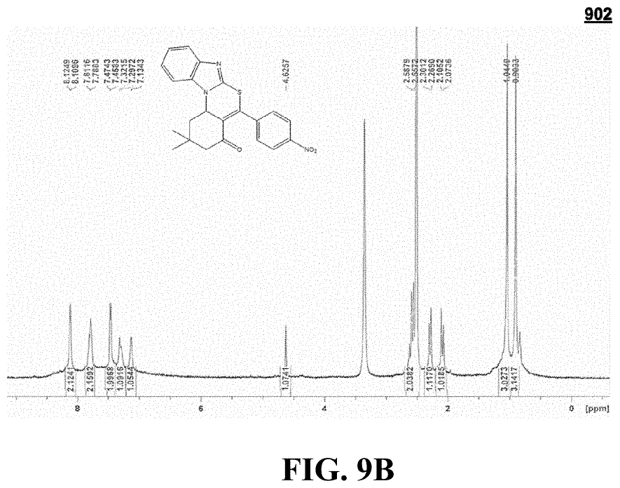

FIG. 9A illustrates exemplary ¹H NMR spectrum 900 of benzo[4,5]imidazo[2,1-b]pyrimido[4,5-d][1,3]thiazine-2,4(3H)-dione, consistent with one or more exemplary embodiments of the present disclosure. ¹H NMR technique may employ nuclear magnetic resonance to identify or confirm an exemplary structure of an organic compound or exemplary compounds that may have protons. In further detail with respect to ¹H NMR spectrum 900, a singlet at about 2.91 ppm may correspond to methyl protons of benzo[4,5]imidazo[2,1-b]pyrimido[4,5-d][1,3]thiazine-2,4(3H)-dione. Singlets at about 3.56 ppm and 3.66 ppm may correspond to N—CH₃ protons in an exemplary structure of in benzo[4,5]imidazo[2,1-b]pyrimido[4,5-d][1,3]thiazine-2,4(3H)-dione. Furthermore, a doublet detected at about 7.69 ppm (J=5.8 Hz), a multiplet at about 7.96-7.97 ppm, a multiplet at about 8.01-8.03 ppm, a doublet at about 8.11 ppm (J=5.65 Hz), and a multiplet at about 8.17-8.23 ppm may correspond to aromatic protons of benzo[4,5]imidazo[2,1-b]pyrimido[4,5-d][1,3]thiazine-2,4(3H)-dione.

FIG. 9B illustrates exemplary ¹H NMR spectrum 902 of benzo[4,5]imidazo[2,1-b][1,3]thiazin-4-one, consistent with one or more exemplary embodiments of the present disclosure. In further detail with respect to ¹H NMR spectrum 902, the detected singlets at about 0.90 ppm and about 1.04 ppm may correspond to CH₃ protons of benzo[4,5]imidazo[2,1-b][1,3]thiazin-4-one. A doublet at about 2.08 ppm (J=15.8 Hz), a doublet at about 2.28 ppm (J=16.1 Hz), a multiplet at about 2.55-2.58 ppm, and a singlet at about 4.62 ppm were observed that may correspond to CH proton. Furthermore, the presence of aromatic protons was confirmed by observing a doublet at about 7.13 ppm (J=6.20), a multiplet about 7.29-7.32 ppm, a doublet at about 7.46 ppm (J=8 Hz), a multiplet at about 7.78-7.81 ppm, and a doublet at about 8.11 ppm (J=7.65 Hz) in ¹H NMR spectrum 902.

### Example 2: Evaluating Anti-Cancer Activity of benzo[4,5]imidazo[2,1-b]pyrimido[4,5-d][1,3]thiazine-2,4(3H)-dione and benzo[4,5]imidazo[2,1-b][1,3]thiazin-4-one

In this example, anti-cancer activity of benzo[4,5]imidazo[2,1-b]pyrimido[4,5-d][1,3]thiazine-2,4(3H)-dione and benzo[4,5]imidazo[2,1-b][1,3]thiazin-4-one was evaluated on A-431 cell line (an exemplary epidermoid carcinoma cell line), PC-3 cell line (an exemplary classical prostate cancer cell line), PANC-1 cell line (an exemplary pancreas ductal adenocarcinoma cell line)) by conducting an exemplary 2,5-diphenyl-2H-tetrazolium bromide (MTT) assay. Cytotoxic effect of benzo[4,5]imidazo[2,1-b]pyrimido[4,5-d][1,3]thiazine-2,4(3H)-dione and benzo[4,5]imidazo[2,1-b][1,3]thiazin-4-one was also evaluated on Human Dermal Fibroblasts (HDF) cell line (an exemplary normal, human, adult cell line).

Meanwhile, the A-431, PC-3, and PANC-1 cell lines were tested by MTT assay after being cultured in DMSO with a concentration of about 1% (v/v) and Etoposide (an anti-cancer chemotherapy drug) as negative and positive controls, respectively. Table 1 below shows IC50 values obtained by MTT assay for benzo[4,5]imidazo[2,1-b]pyrimido[4,5-d][1,3]thiazine-2,4(3H)-dione, benzo[4,5]imidazo[2,1-b][1,3]thiazin-4-one, and Etoposide after 24 hours of treatment. “IC50” may refer to a concentration of a compound that may inhibit cell growth and proliferation by 50%. As shown in Table 1, IC50 values of benzo[4,5]imidazo[2,1-b]pyrimido[4,5-d][1,3]thiazine-2,4(3H)-dione and benzo[4,5]imidazo[2,1-b][1,3]thiazin-4-one was significantly higher than IC50 values of Etoposide.

TABLE 1
IC50 values obtained by MTT assay for benzo[4,5]imidazo[2,1-
b]pyrimido[4,5-d][1,3]thiazine-2,4(3H)-dione,
benzo[4,5]imidazo[2,1-b][1,3]thiazin-4-one,
and Etoposide after 24 hours of treatment, consistent
with one or more exemplary embodiments of the present disclosure.

| | IC50 (μM) | | | |
|---|---|---|---|---|
| Compound | A-431 | PANC-1 | PC-3 | HDF |
| benzo[4,5]imidazo[2,1- | 25.33 + | 24.59 + | 31.87 + | >100 |
| b]pyrimido[4,5-d][1,3]thiazine- | 0.017 | 0.028 | 0.025 | |
| 2,4(3H)-dione | | | | |
| benzo[4,5]imidazo[2,1- | 22.15 + | 21.57 + | 21.59 + | >100 |
| b][1,3]thiazin-4-one | 0.019 | 0.02 | 0.017 | |
| Etoposide | 27.01 + | 32.18 + | 25.19 + | >100 |
| | 0.021 | 0.24 | 0.019 | |

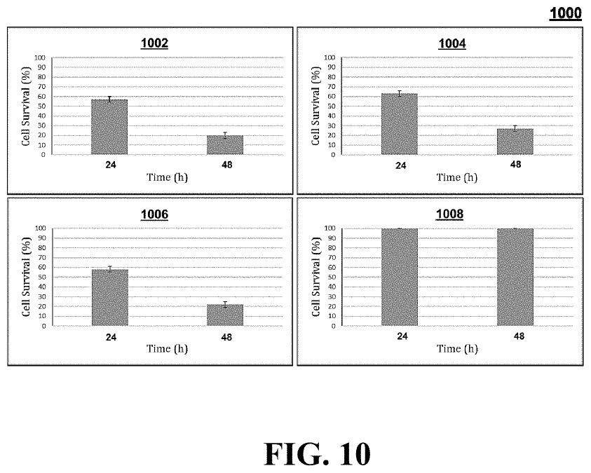

FIG. 10 illustrates charts 1000 of cell survival assessment after 24 hours of treatment with benzo[4,5]imidazo[2,1-b][1,3]thiazin-4-one, consistent with one or more exemplary embodiments of the present disclosure. In further detail with respect to charts 1000, chart 1002 shows the survival rate of PC-3 cell after treatment with benzo[4,5]imidazo[2,1-b][1,3]thiazin-4-one for 24 and 48 hours, chart 1004 shows the survival rate of PANC-1 cells after treatment with benzo[4,5]imidazo[2,1-b][1,3]thiazin-4-one for 24 and 48 hours, chart 1006 shows the survival rate of A-431 cells after treatment with benzo[4,5]imidazo[2,1-b][1,3]thiazin-4-one for 24 and 48 hours, and chart 1008 shows the survival rate of HDF cells after treatment with benzo[4,5]imidazo[2,1-b][1,3]thiazin-4-one for 24 and 48 hours. The obtained IC50 values (shown in Table 1) and the survival rates of PC-3 cell line (chart 1002), PANC-1 cell line (chart 1004), and A-431 cell line (chart 1006) that were cultured in the presence of benzo[4,5]imidazo[2,1-b][1,3]thiazin-4-one demonstrate anti-cancer effect of benzo[4,5]imidazo[2,1-b][1,3]thiazin-4-one on one skin, prostate, and pancreatic cancer cells.

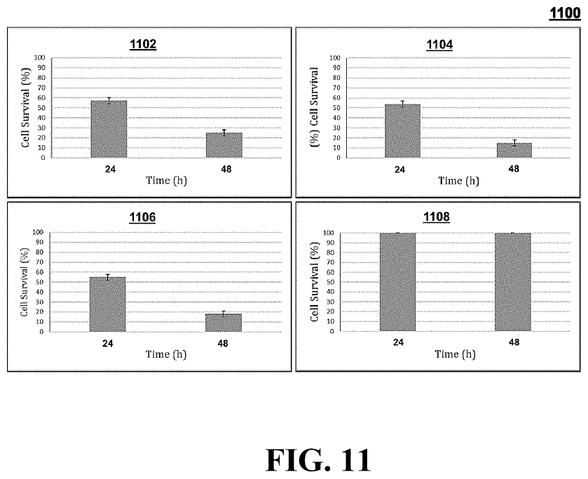

FIG. 11 illustrates charts 1100 of cell survival assessment after 24 hours of treatment with benzo[4,5]imidazo[2,1-b]pyrimido[4,5-d][1,3]thiazine-2,4(3H)-dione, consistent with one or more exemplary embodiments of the present disclosure. In further detail with respect to charts 1100, chart 1102 shows the survival rate of PC-3 cell after treatment with benzo[4,5]imidazo[2,1-b]pyrimido[4,5-d][1,3]thiazine-2,4(3H)-dione for 24 and 48 hours, chart 1104 shows the survival rate of PANC-1 cells after treatment with benzo[4,5]imidazo[2,1-b]pyrimido[4,5-d][1,3]thiazine-2,4(3H)-dione for 24 and 48 hours, chart 1106 shows the survival rate of A-431 cells after treatment with benzo[4,5]imidazo[2,1-b]pyrimido[4,5-d][1,3]thiazine-2,4(3H)-dione for 24 and 48 hours, and chart 1108 shows the survival rate of HDF cells after treatment with benzo[4,5]imidazo[2,1-b]pyrimido[4,5-d][1,3]thiazine-2,4(3H)-dione for 24 and 48 hours. The obtained IC50 values (shown in Table 1) and the survival rates of PC-3 cell line (chart 1102), PANC-1 cell line (chart 1104), and A-431 cell line (chart 1106) that were cultured in the presence of benzo[4,5]imidazo[2,1-b]pyrimido[4,5-d][1,3]thiazine-2,4(3H)-dione demonstrate anti-cancer effect of benzo[4,5]imidazo[2,1-b]pyrimido[4,5-d][1,3]thiazine-2,4(3H)-dione on on skin, prostate, and pancreatic cancer cells.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. An element proceeded by “a” or “an” does not, without further constraints, preclude the existence of additional identical elements in the process, method, article, or apparatus that comprises the element.

Unless otherwise stated, all measurements, values, ratings, positions, magnitudes, sizes, and other specifications that are set forth in this specification, are approximate, not exact. They are intended to have a reasonable range that is consistent with the functions to which they relate and with what is customary in the art to which they pertain.

It will be understood that the terms and expressions used herein have the ordinary meaning as is accorded to such terms and expressions with respect to their corresponding respective areas of inquiry and study, except where specific meanings have otherwise been set forth herein. Relational terms such as “first” and “second” and the like may be used solely to distinguish one entity or action from another without necessarily requiring or implying any actual such relationship or order between such entities or actions.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it may be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A method for synthesizing a compound comprising the following structural formula (I): 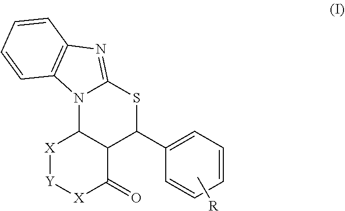
wherein:
X is selected from the group consisting of NCH₃ and CH₂;
Y is selected from the group consisting of C═O and C(CH₃)₂; and
R is selected from the group consisting of 4-CH₃ and 4-NO₂;
the method comprising:
forming a liquid mixture by adding a CH-acid, 2-Mercaptobenzimidazole, and a benzaldehyde component to an ionic liquid of trihexyltetradecylphosphonium bromide, wherein the liquid mixture comprises:
trihexyltetradecylphosphonium bromide with a concentration between 47% (w/w) and 53% (w/w);
the CH-acid selected from the group consisting of 1,3-Dimethylbarbituric acid and Dimedone, wherein the CH-acid has a concentration between 15.3% (w/w) and 21.3% (w/w);
2-Mercaptobenzimidazole with a concentration between 14.5% (w/w) and 20.6% (w/w); and
the benzaldehyde component selected from the group consisting of 4-Methylbenzaldehyde and 4-Nitrobenzaldehyde, wherein the benzaldehyde component has a concentration between 11.5% (w/w) and 17.5% (w/w);
mixing the liquid mixture at a temperature between 20° C. and 30° C. for a time duration between 17 and 30 minutes; and
forming the compound by adding an ethanol-water solution to the mixed liquid mixture with a volume ratio (ethanol-water solution:mixed liquid mixture) between 1:1 and 1.5:1, wherein the ethanol-water solution comprises ethanol with a concentration between 30% (v/v) and 45% (v/v).

2. A method for synthesizing a compound comprising the following structural formula (I): 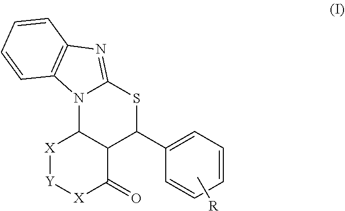
wherein:
X is selected from the group consisting of NCH₃ and CH₂;
Y is selected from the group consisting of C═O and C(CH₃)₂; and
R is selected from the group consisting of 4-CH₃ and 4-NO₂;
the method comprising:
forming a liquid mixture by adding a CH-acid, 2-Mercaptobenzimidazole, and a benzaldehyde component to an ionic liquid of trihexyltetradecylphosphonium bromide, wherein:
the CH-acid is selected from the group consisting of 1,3-Dimethylbarbituric acid and Dimedone; and
the benzaldehyde component is selected from the group consisting of 4-Methylbenzaldehyde and 4-Nitrobenzaldehyde;
mixing the liquid mixture at a temperature between 20° C. and 30° C. for a time duration between 17 and 30 minutes; and
forming the compound by adding an ethanol-water solution to the mixed liquid mixture with a volume ratio (ethanol-water solution:mixed liquid mixture) between 1:1 and 1.5:1, wherein the ethanol-water solution comprises ethanol with a concentration between 30% (v/v) and 45% (v/v).

3. The method of claim 2, wherein the liquid mixture consists of the trihexyltetradecylphosphonium bromide, the CH-acid, the 2-Mercaptobenzimidazole, and the benzaldehyde component with a molar ratio (trihexyltetradecylphosphonium bromide:CH-acid:2-Mercaptobenzimidazole:benzaldehyde component) of 1:1:1:1.

4. The method of claim 2, wherein the trihexyltetradecylphosphonium bromide has a concentration between 47% (w/w) and 53% (w/w).

5. The method of claim 4, wherein the trihexyltetradecylphosphonium bromide has a concentration between 49% (w/w) and 51% (w/w).

6. The method of claim 2, wherein the CH-acid has a concentration between 15.3% (w/w) and 21.3% (w/w).

7. The method of claim 6, wherein the CH-acid has a concentration between 16% (w/w) and 18.5% (w/w).

8. The method of claim 2, wherein the 2-Mercaptobenzimidazole has a concentration between 14.5% (w/w) and 20.6% (w/w).

9. The method of claim 8, wherein the 2-Mercaptobenzimidazole has a concentration between 17% (w/w) and 18% (w/w).

10. The method of claim 2, wherein the benzaldehyde component has a concentration between 11.5% (w/w) and 17.5% (w/w).

11. The method of claim 10, wherein the benzaldehyde component has a concentration between 14% (w/w) and 17.5% (w/w).

12. The method of claim 2, wherein forming the liquid mixture comprises forming the liquid mixture comprising:
the trihexyltetradecylphosphonium bromide with a concentration between 47% (w/w) and 53% (w/w);
the CH-acid with a concentration between 15.3% (w/w) and 21.3% (w/w);
the 2-Mercaptobenzimidazole with a concentration between 14.5% (w/w) and 20.6% (w/w); and
the benzaldehyde component with a concentration between 11.5% (w/w) and 17.5% (w/w).
